(19) United States
(12) Patent Application Publication
(10) Pub. No.: US 2026/0096993 A1
(43) Pub. Date: Apr. 9, 2026

(54) Effervescent tablet

(71) Applicants: Philip Mackenzie, Oakville (CA); Dan Aziz, Boston, MA (US)

(72) Inventors: Philip Mackenzie, Oakville (CA); Dan Aziz, Boston, MA (US)

(21) Appl. No.: 18/909,065

(22) Filed: Oct. 8, 2024

(51) Int. Cl. A61K 9/20; A61K 9/46

(52) U.S. Cl. CPC A61K 9/2072; A61K 9/0007

(57) ABSTRACT

Disclosed is a teardrop shaped tablet defined in a top or bottom plan by two asymmetric and eccentric circles within a reference circle. Top and bottom surfaces are cupped and the sidewalls are upright. The sidewalls and top surfaces meet with a blended land. This design allows the tablet to be inserted into the vessel, enhancing its ability to dissolve quickly and efficiently. The overall thickness, curvature, and other geometric properties are optimized to ensure rapid dissolution, making the tablet suitable for delivering nutrients or medications that are difficult to digest or require large doses. The invention aims to improve the bioavailability of active ingredients, making them more effective for certain nutritional or medical needs, and represents a significant advancement in the field of effervescent tablets.

### CROSS-REFERENCE TO RELATED APPLICATIONS

See application data sheet (ADS). if applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of effervescent tablets.

### Background of the Invention

Effervescent tablets date back to the late-nineteenth and early-twentieth centuries. Effervescent tablets are a mixture of powders that violently react when submerged in water such that a powdered nutrient or medication quickly dissolve in the water. More specifically, carbon dioxide (CO2) is released, and dissolution of the nutrient or medication is enhanced when the water exposure triggers a superficial (e.g., on the tablet's surface) reaction between an acid (e.g., citric acid) and a bicarbonate (such as sodium bicarbonate).

One of the earliest effervescent tablets was branded as Aka-Seltzer® in 1931. Alka-Seltzer® combined aspirin, sodium bicarbonate and anhydrous citric acid in a tablet. The situation enables a large dose of aspirin and baking soda could quickly be dissolved in a cup of water to treat the flu, heartburn and indigestion.

Presently, effervescent tablets are widely used in the pharmaceutical and dietary industries for their rapid dissolution and absorption properties. They are said to be particularly beneficial for delivering nutrients or medications that are difficult to digest or require large doses. The effervescent format enhances the bioavailability of active ingredients, making them more effective for certain nutritional or medical needs.

Effervescent tablets have been around for a century without many improvements. For example, the powder forming Alka-Seltzer® and other known effervescent tablets is still basically stamped into a disk at the same density and surface area as the original tablet formulation. Suitably, the tablet's density is thought to be optimized for triggering the superficial reaction and dissolution of the active ingredient in water. Additionally, the disk-shape is thought to be chosen for easy manufacture and packaging. Often, the is disk is scored so that the tablet may, at the option of the user, be easily broken (e.g., in half) to reduce the dose, increase the surface area of the tablet for faster dissolution, or decrease a dimension of the tablet for insertion into a water-containing vessel with a small opening.

### SUMMARY OF THE INVENTION

In view of the foregoing, an object of this specification is to disclose an improved effervescent tablet and related methods of dissolving supplements or medicines in water in a bottle or other vessel. The disclosure focuses on enhancing the design and functionality of effervescent tablets, which are used to dissolve nutrients or medications in water. Traditionally, these tablets have been in a disk shape, but the Applicant's new design introduces a teardrop-shaped tablet. This size and shape of the design are specifically engineered to facilitate easy insertion into bottles or vessels with small openings and to improve the dissolution process of the active ingredients. The tablet's design includes upright sidewalls and cupped top and bottom surfaces, which aid in its dissolution properties. The dimensions are carefully calculated, with the tablet weighing approximately 4,000 mg, and specific tolerances are applied to ensure consistency and effectiveness.

The teardrop shape is defined by two asymmetric and eccentric circles within a reference circle, with specific dimensions provided for each component of the shape. The effervescent tablet is a teardrop shape by top or bottom plan. Suitably, the teardrop shape is defined by eccentric circles. The sidewalls of the tablet are upright and the top and bottom surfaces are cupped. The top and bottom surfaces meet the sidewalls with a blended land to form a corner and edge. This design allows the tablet to be inserted into the vessel, enhancing its ability to dissolve quickly and efficiently. The overall thickness, curvature, and other geometric properties are optimized to ensure rapid dissolution, making the tablet suitable for delivering nutrients or medications that are difficult to digest or require large doses. The invention aims to improve the bioavailability of active ingredients, making them more effective for certain nutritional or medical needs, and represents a significant advancement in the field of effervescent tablets.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is perspective view of an effervescent tablet; and,

FIG. 2 is another perspective view of the effervescent tablet;

FIG. 3 is a top or bottom plan view of the effervescent tablet

FIG. 4 is a front view of the effervescent tablet;

FIG. 5 is a side view of the effervescent tablet;

FIG. 6 is top or bottom dimensional view of the effervescent tablet;

FIG. 7 is a side, dimensional view of the effervescent tablet;

FIG. 8 is a front view of the alternative embodiment of the effervescent tablet;

FIG. 9 is a detail view of circle 9 of FIG. 8; and,

FIG. 10 is an environmental view of the effervescent tablet.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFFERED EMBODIMENTS

Disclosed is and improved effervescent tablet and related methods of use.

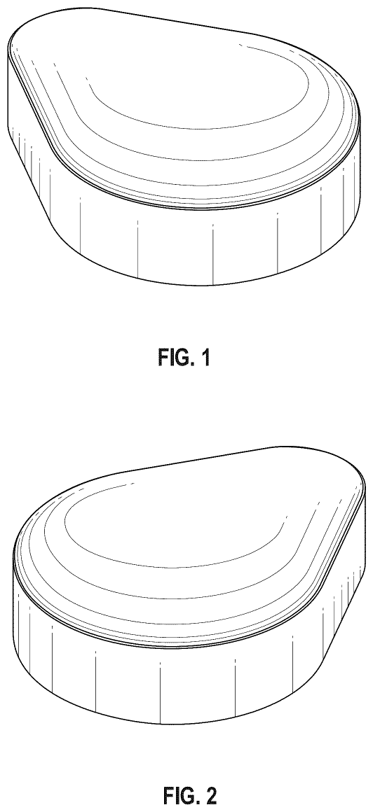

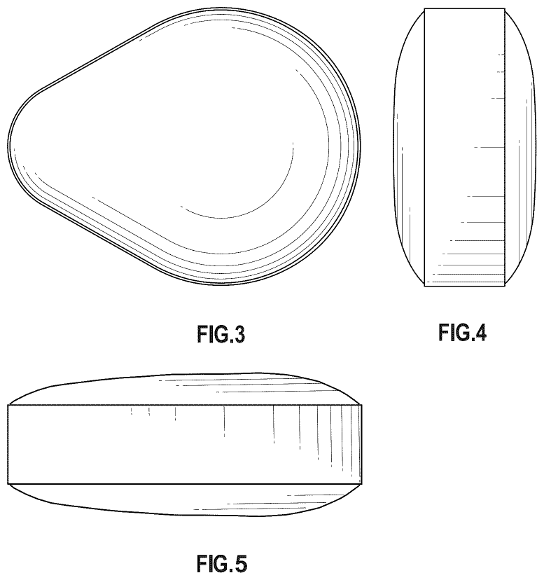

FIGS. 1 and 2 are perspective views of an effervescent tablet. FIGS. 3 through 5 are respectively a front view, a side view and a top or bottom view of the effervescent tablet. As shown, the tablet is teardrop shaped and, as discussed in greater detail below, the tablet has properties for easy (a) insertion into water within a vessel with a small opening and (b) dissolution of a medicine or nutrient supplement in water.

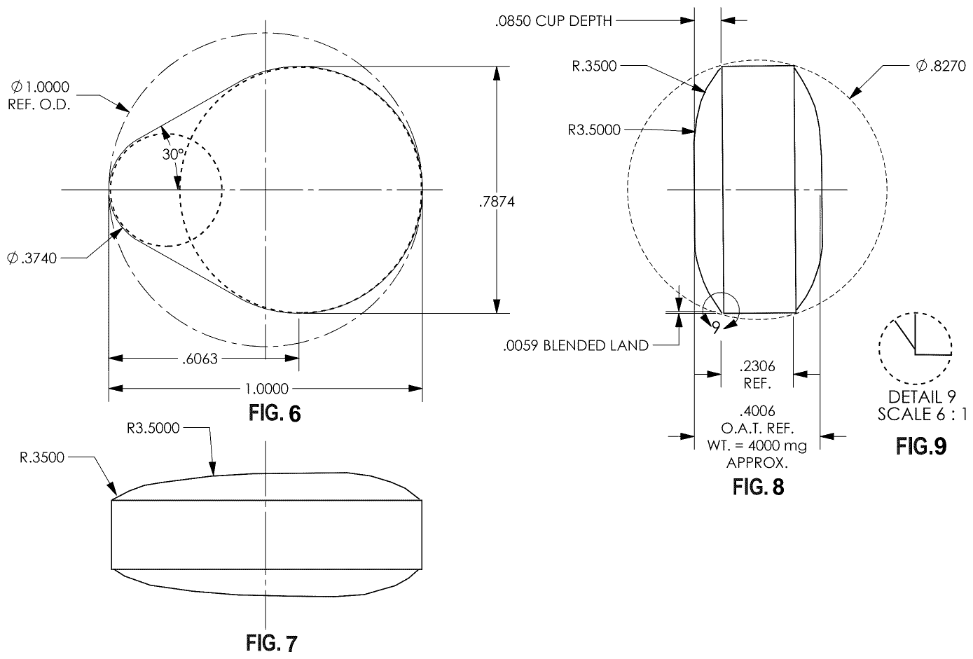

FIG. 6 is top or bottom dimensional view of the effervescent tablet. As shown, the plan of the tablet is a teardrop shape defined by two, asymmetric and eccentric circles within a reference circle. The diameter of a first one of the eccentric circles is 0.3740 inches. The diameter of the second one of the eccentric circles is 0.7874 inches. A diameter of the reference circle that contains the two eccentric circles is 1.0000 inch. The distance from the tip of the teardrop to the center of the second one of the eccentric circles is 0.6063 inches. Additionally, the teardrop shape is 1.00 inch tall and 0.7874 inches wide in view of the circular dimensions mentioned above. The outer surface of the teardrop is defined by a surface that is tangent to both circles such that the angle formed by the tangent surface and a centerline of the eccentric circles is thirty degrees relative to the centerline.

FIGS. 7 and 8 are respectively a side dimensional view and a front dimensional view of the effervescent tablet. As shown, the tablet features upright side walls that are 0.2306 inches thick. The top and bottom surfaces of the tablet are cupped with a cup depth of 0.0850 inches such that the overall thickness of the tablet features an 0.4006 inch overall assembly tolerance reference. As further shown, the radius of curvature for the cup is a blend of 0.3500 inches and 3.5000 inches. Referring to FIG. 8, the front of the tablet may be fit within a reference circle that has a diameter of 0.8270 inches.

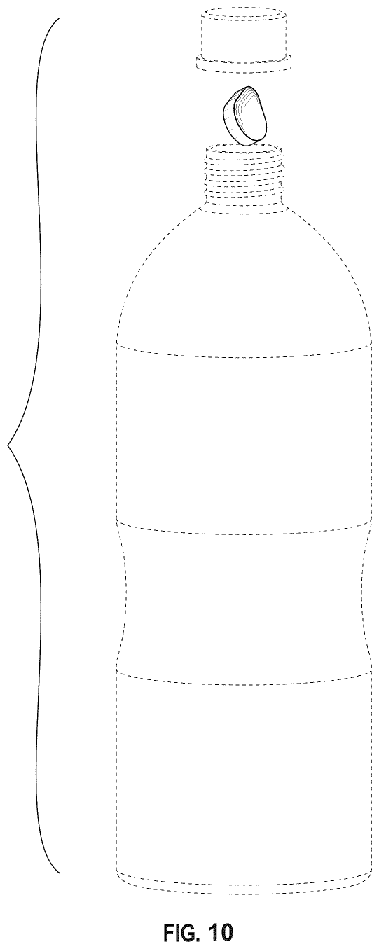

FIG. 9 is a detail view of circle 9 of FIG. 8A. This detailed view shows the perimeter corner and edge of the tablet that is implied by the difference between the diameter of the 0.8270 inch reference circle of FIG. 8 and the second eccentric circle of FIG. 6. The corner or edge is sometimes referred to a blended land that is 0.059 inches wide (as set forth in FIG. 8).

Referring to FIGS. 6 through 9, the weight of the tablet may be approximately 4000 mg, with this weight being based on 21,000 mg/in³ compressed density. Tolerances of the dimensions shown in the figures are plus or minus 0.0762 millimeters (mm) for decimal tolerances or an angular tolerance of plus or minus 0.33 degrees. Additionally, the dimensions, weights and other properties of the tablet reflected in these figures should be considered nominal tool sizes that do not account for tablet shrinkage (contraction) or expansion (enlargement). The tooling size data has an upper tip size of 0.4825 inch×0.4968 inch and a lower tip size of 0.4290 inch×0.4973 inch plus 0.0000 inches or minus 0.0005 inches. Suitably, the die size may be 0.7874 inch×1.0000 inch plus 0.0005 inches or minus 0.0000 inches. Finally, the cup volume may suitably be 0.02928 in³, the cup area may be 0.59532 in², the perimeter may be 2.76706 inches, and the die hole surface area may be 0.56600 in².

FIG. 10 is an environmental view of the effervescent tablet. As shown, the tablet may be deposited into a water containing vessel such that nutrients or medication may be dissolved in the water. Suitably, the tip of the teardrop defined by the first of the two eccentric circles in the top view of FIG. 6 may be inserted last into the opening of the vessel such that the teardrop may enter the vessel like a drop and assist in penetration of the opening via the tablet.

Although the method and apparatus is described above in terms of various exemplary embodiments and implementations, it should be understood that the various features, aspects, and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus, the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more,” or the like; and adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to,” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A teardrop-shaped effervescent tablet, characterized by its design with two asymmetric and eccentric circles within a reference circle, which facilitates easy insertion into vessels with small openings and enhances the dissolution of nutrients or medications in water.

2. An effervescent tablet featuring upright sidewalls and cupped top and bottom surfaces, where the top and bottom surfaces have a cup depth of 0.0850 inches and a blend of curvature radii of 0.3500 inches and 3.5000 inches, optimizing the tablet for rapid dissolution in water.

3. A method of constructing an effervescent tablet comprising the steps of:
Securing a nutrient, an anhydrous acid, and a bicarbonate with a combined weight of approximately 4000 mg, constructed with a compressed density of 21,000 mg/in³, and designed with specific dimensional tolerances to ensure consistency and effectiveness in dissolving active ingredients in water.
